(19) United States
(12) Patent Application Publication
(10) Pub. No.: US 2025/0191139 A1
(43) Pub. Date: Jun. 12, 2025

(54) SYSTEMS AND METHODS FOR CONTRAST-ENHANCED MRI

(71) Applicant: Subtle Medical, Inc., Menlo Park, CA (US)

(72) Inventors: Jonathan TAMIR, Menlo Park, CA (US); Srivathsa PASUMARTHI VENKATA, Sunnyvale, CA (US); Enhao GONG, Sunnyvale, CA (US)

(21) Appl. No.: 19/058,491

(22) Filed: Feb. 20, 2025

Related U.S. Application Data
(63) Continuation of application No. PCT/US2023/072081, filed on Aug. 11, 2023.
(60) Provisional application No. 63/400,307, filed on Aug. 23, 2022.

(51) Int. Cl. G06T 5/60; G06T 5/50; G06T 5/70

(52) U.S. Cl. CPC G06T 5/60; G06T 5/50; G06T 5/70; G06T 2207/10088; G06T 2207/20081; G06T 2207/20084; G06T 2207/20212; G06T 2207/30004

(57) ABSTRACT

Methods and systems are provided for improving image quality without increasing dose of contrast agent. The method comprises: (a) receiving an input image comprising a pre-contrast image and a full-dose image, the pre-contrast image is a volumetric medical image of a subject acquired without administering contrast agent and the full-dose image is a volumetric image of the subject acquired with standard dose of contrast agent; (b) selecting a path from a plurality of paths to process the input image, the path comprises at least a first model trained to predict a contrast-enhanced image, and a second model trained to denoise an image and wherein the first model and the second model are arranged in a predetermined order to process the input image; generating a predicted image by processing the input image using the path selected in (b), the predicted image has an image quality improved over the input image.

### CROSS-REFERENCE TO RELATED APPLICATION

This application is a continuation of International Application No. PCT/US2023/072081 filed on Aug. 11, 2023, which claims priority to U.S. Provisional Application No. 63/400,307 filed on Aug. 23, 2022, the content of which is incorporated herein in its entirety.

### BACKGROUND

Contrast agents such as Gadolinium-based contrast agents (GBCAs) have been used in approximately one third of Magnetic Resonance imaging (MRI) exams worldwide to create indispensable image contrast for a wide range of clinical applications. Despite the use of contrast agent, the MRI imaging quality may still not be satisfying. Deep learning technique has been used in volumetric contrast-enhanced MRI, but challenges in generalizability remain due to variability in scanner hardware and clinical protocols within and across sites.

### SUMMARY

The present disclosure provides improved imaging systems and methods that can address various drawbacks of conventional systems, including those recognized above. Methods and systems as described herein can improve quality of images that are acquired with contrast agent such as Gadolinium-Based Contrast Agents (GBCAs). In particular, a generalized deep learning (DL) model is utilized to predict contrast-enhanced images across different sites and scanners.

Traditionally, contrast agent such as Gadolinium-Based Contrast Agents (GBCAs) and others has been used in a wide range of contrast-enhanced medical imaging such as Magnetic Resonance Imaging (MRI), or nuclear magnetic resonance imaging, for examining pathology, predicting prognosis and evaluating treatment response for gliomas, multiple sclerosis (MS), Alzheimer's disease (AD), and the like. GBCAs are also pervasive in other clinical applications such as evaluation of coronary artery disease (CAD), characterization of lung masses, diagnosis of hepatocellular carcinoma (HCC), imaging of spinal metastatic disease. To achieve higher image quality, either the dose level of Gadolinium is increased which can result in an increase of the contrast agent retention in the subject body, or the scan time is prolonged which can result in an increase of patient inconvenience during intravenous injection and an overall increase in imaging costs. Even though GBCAs have a good pharmacovigilance safety profile, there is a clear need for improving the image quality without further increasing the dose level due to the abovementioned safety issues and concerns. In particular, it is desirable to provide a safe imaging technique where the image quality can be enhanced without extra contrast dose.

Recent developments in Deep learning (DL) or machine learning (ML) techniques enable it as a potential alternative to the excessive use of contrast dose. Although deep learning models may be able to reduce dose levels while maintaining non-inferior image quality, the DL enhanced images often suffer from artifacts such as streaks on a reformat image (e.g., reformatted volumetric image or reconstructed 3D image viewed from different planes, orientations or angles).

There exists a need for providing a robust DL model that is generalized for (sometimes agnostic to) diverse clinical settings such as different scanner vendors, scan protocols, patient demographics, and clinical indications. Such a model is also desired to produce artifact-free images and support a variety of clinical use cases such as multiplanar reformat (MPR) for oblique visualizations of 3D images, thus enabling the model to be deployed and integrated within a standard clinical workflow.

Systems and methods described herein can address the abovementioned drawbacks of the conventional solutions. In particular, the provided systems and methods may involve a DL model including a unique set of algorithms and methods that improve the model robustness and generalizability. The algorithms and methods may include, for example, multi-planar reconstruction, 2.5D deep learning model, enhancement-weighted L1, perceptual and adversarial losses algorithms and methods, as well as pre-processing algorithms that are used to pre-process the input pre-contrast (e.g., images acquired without contrast agent) and full-dose images (e.g., images acquired with full-dose level of contrast agent) prior to the model predicting the corresponding contrast-enhanced images. The term “contrast-enhanced images” as utilized herein, may generally refer to a synthesized image generated using the methods herein that mimic an image acquired with increased dose of contrast agent. For instance, a synthesized image produced by a contrast boost deep learning model herein may have an image quality enhanced over the image quality of the input image, or the quality of the synthesized image may be same as an image acquired with a higher contrast agent dose compared to the contrast agent dose administered to the input image.

The DL model that is trained to boost contrast (i.e., predicting contrast-enhanced images) can be utilized in combination with other models that are trained to denoise the image, and/or synthesize super-resolution image. The methods and systems of the present disclosure beneficially provide various combination of the models and mechanism. In some cases, the images may be processed by a first model (e.g., contrast boost model) trained to predict contrast-enhanced image, a second model trained to denoise the image, and a third model to improve the resolution of the image. The images may be processed in a selected processing path that comprises a combination of the above models in a selected order. In some cases, a processing path may comprise repeatedly applying one or more of the models. In some embodiments, the processing path may comprise multi-contrast branched architecture combined with the contrast-boost model. In some cases, a processing path may be selected based on the quality of the input image, the use application (e.g., anomaly detection), user preference, the subject being imaged (e.g., organ, tissue), or any other conditions.

In an aspect, a method is provided for improving image quality without increasing dose of contrast agent. The method comprises: (a) receiving an input image comprising a pre-contrast image and a full-dose image, the pre-contrast image is a volumetric medical image of a subject acquired without administering contrast agent and the full-dose image is a volumetric image of the subject acquired with standard dose of contrast agent; (b) selecting a path from a plurality of paths to process the input image, the path comprises at least a first model trained to predict a contrast-enhanced image, and a second model trained to denoise an image and wherein the first model and the second model are arranged in a predetermined order to process the input image; and generating a predicted image by processing the input image using the path selected in (b), the predicted image has an image quality improved over the input image.

In a related yet separate aspect, a non-transitory computer-readable storage medium including instructions that, when executed by one or more processors, causes the one or more processors to perform operations. The operations comprise: (a) receiving an input image comprising a pre-contrast image and a full-dose image, the pre-contrast image is a volumetric medical image of a subject acquired without administering contrast agent and the full-dose image is a volumetric image of the subject acquired with standard dose of contrast agent; (b) selecting a path from a plurality of paths to process the input image, the path comprises at least a first model trained to predict a contrast-enhanced image, and a second model trained to denoise an image and wherein the first model and the second model are arranged in a predetermined order to process the input image; and generating a predicted image by processing the input image using the path selected in (b), the predicted image has an image quality improved over the input image.

In some embodiments, the path further comprises a third model trained to improve a resolution of an image. In some cases, each of the plurality of paths comprises two or more of the first model, the second model and the third model arranged in a predetermined order.

In some embodiments, the input image further comprises one or more reformatted volumetric medical images of the pre-contrast image or the full-dose image. In some cases, the one or more reformatted volumetric medical images are generated by reformatting the pre-contrast image or the full-dose image in one or more orientations. In some instances, the one or more orientations include at least one orientation that is not in a direction of a scanning plane.

In some embodiments, at least one of the plurality of paths comprises two of the second models to denoise the pre-contrast image and the full-dose image respectively.

In some embodiments, the pre-contrast image or the full-dose image is acquired using a transforming magnetic resonance (MR) device. In some cases, the input image comprises different contrast-weighted images acquired using different pulse sequences. In some instances, the different contrast-weighted images comprise two or more selected from the group consisting of T1-weighted (T1), T2-weighted (T2), proton density (PD) or Fluid Attenuation by Inversion Recovery (FLAIR). In some examples, at least one of the plurality of paths comprise a multi-contrast branched architecture. In some cases, the multi-contrast branched architecture comprises multiple branches and wherein inputs to the multiple branches are different in at least one of dose of contrast agent and pulse sequence. For instance, each of the multiple branches comprises a first model trained to learn features of the respective input image. In some instances, a plurality of synthesized images generated by the multiple branches are aggregated and is further processed by a trained model generate a final output image.

Additionally, methods and systems of the present disclosure may be applied to existing systems without a need of a change of the underlying infrastructure. In particular, the provided methods and systems may enhance the image quality at no additional cost of hardware component and can be deployed regardless of the configuration or specification of the underlying infrastructure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a workflow for processing and reconstructing magnetic resonance imaging (MRI) volumetric image data.

FIG. 2 shows an example of data collected from the two different sites.

FIG. 3 shows the analytic results of a study to evaluate the generalizability and accuracy of the contrast boost model.

FIG. 4 schematically illustrates a magnetic resonance imaging (MRI) system in which an imaging enhancer of the presenting disclosure is implemented.

FIG. 5 schematically shows an example of processing input images including pre-contrast image and full-dose image with the trained contrast boost model.

FIG. 6 illustrates an example of a reformat MPR reconstructed image that have a quality improved over the reformat MRI image generated using a conventional method.

FIG. 7 shows an example of a pre-processing method, in accordance with some embodiments herein.

FIG. 8 shows an example of a U-Net style encoder-decoder network architecture, in accordance with some embodiments herein.

FIG. 9 shows an example of the discriminator, in accordance with some embodiments herein.

FIG. 10 shows examples of contrast enhanced images outputted by a contrast boost model.

FIG. 11 shows examples of pre-contrast, low-dose, full-dose ground truth image data and synthesized images along with the quantitative metrics for cases from different sites and scanners.

FIG. 12 shows exemplary processing paths including various combinations of a contrast boost model and a denoise model.

FIG. 13 and FIG. 14 show examples of the output image generated by different processing paths in FIG. 12.

FIG. 15 shows multiple exemplary processing paths comprising various combinations of a contrast boost model and a super-resolution model.

FIG. 16 and FIG. 17 show examples of the output image generated by the different processing paths in FIG. 15.

FIG. 18 shows an exemplary processing path comprising a combination of a denoise model, a contrast boost model, and a resolution model.

FIG. 19 shows an example of a processing path comprising a multi-contrast branched architecture.

FIG. 20 schematically illustrates a magnetic resonance imaging (MRI) system in which an imaging enhancer of the presenting disclosure may be implemented.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Gadolinium-based contrast agents (GBCAs) are widely used in magnetic resonance imaging (MRI) exams and have been indispensable for monitoring treatment and investigating pathology in myriad applications including angiography, multiple sclerosis and tumor detection. Recently, the identification of prolonged gadolinium deposition within the brain and body has raised safety concerns about the usage of GBCAs. Increasing the GBCA dose can improve contrast enhancement and tumor conspicuity but at the cost of high risk of gadolinium deposition.

Though MRI, Gadolinium-based contrast agents, MRI data examples are primarily provided herein, it should be understood that the present approach can be used in other imaging modality contexts and/or other contrast-enhanced imaging. For instance, the presently described approach may be employed on data acquired by other types of tomographic scanners including, but not limited to, computed tomography (CT), single photon emission computed tomography (SPECT) scanners, Positron Emission Tomography (PET), functional magnetic resonance imaging (fMRI), or various other types of imaging scanners or techniques wherein a contrast agent may be utilized for enhancing the contrast.

Deep learning (DL) framework has been used to enhance image quality and contrast enhancement for volumetric MRI. As an example, a DL model may use a U-net encoder-decoder architecture to enhance the image contrast from an input image. However, the conventional DL models may only work well with scans from a single clinical site without considering generalizability to different sites with different clinical workflows. Moreover, the conventional DL models may evaluate image quality for individual 2D slices in the 3D volume, even though clinicians frequently require volumetric images to visualize complex 3D enhancing structures such as blood vessels and tumors from various angles or orientations.

The present disclosure provides systems and methods that can address various drawbacks of conventional systems, including those recognized above. Methods and systems of the presenting disclosure capable of improving model robustness and deployment in real clinical settings. For instance, the provided methods and systems are capable of adapting to different clinical sites, each with different MRI scanner hardware and imaging protocols. In addition, the provided methods and systems may provide improved performance while retaining multi-planar reformat (MPR) capability to maintain the clinician workflow and enable oblique visualizations of the complex enhancing microstructure. In some embodiments, the quality of the output image can be further enhanced by selecting a combination of variable types of DL models trained for different tasks.

Methods and systems herein may provide enhancements to the DL model to tackle real-world variability in clinical settings. The DL model is trained and tested on patient scans from different hospitals across different MRI platforms with different scanning planes, scan times, and resolutions, and with different mechanisms for administering GBCA. The robustness of the DL models may be improved in these settings with improved generalizability across a heterogeneity of data.

### Multi-Planar Reformat (MPR)

In a conventional DL pipeline, 2D slices from the 3D volume may be separately processed and trained with standard 2D data augmentation (e.g., rotations and flips). The choice of a 2D model is often motivated by memory limitations during training, and performance requirements during inference. In some cases, DL framework may process the data in a “2.5D” manner, in which multiple adjacent slices are input to a network and the central slice is predicted. However, both 2D and 2.5D processing may neglect the true volumetric nature of the acquisition. As the 3D volume is typically reformatted into arbitrary planes during the clinical workflow (e.g., oblique view, views from orientations/angles that are oblique to the scanning plane/orientation), and sites may use a different scanning orientation as part of their MRI protocol, 2D processing can lead to images with streaking artifacts in the reformat volumetric images (e.g., reformat into planes that are orthogonal to the scanning plane).

Methods and systems described herein may beneficially eliminate the artifacts (e.g., streaking artifacts) in reformat images thereby enhancing the image quality with reduced contrast dose. As described above, reformatting a 3D volume image to view the image in multiple planes (e.g., orthogonal or oblique planes) is common in a standard clinical workflow. In some cases, though training a model to enhance the 2.5D image may reduce the streaking artifacts in the plane of acquisition, reformatting to other orientations may still cause streaking artifacts. Methods and systems as described herein may enable artifact-free visualizations in any selected plane or viewing direction (e.g., oblique view). Additionally, the model may be trained to learn intricate or complex 3D enhancing structures such as blood vessels or tumors.

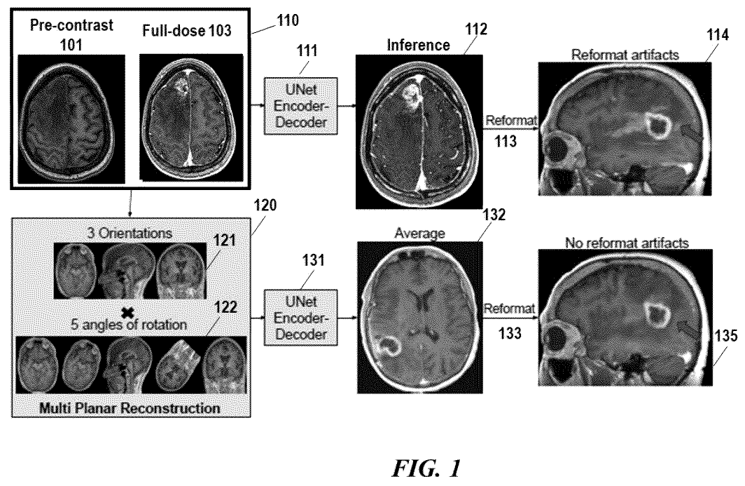

FIG. 1 shows an example of a workflow for processing and reconstructing MRI volumetric image data. As illustrated in the example, the input image 110 may be image slices that are acquired without contrast agent (e.g., pre-contrast image slice 101) and/or with full contrast dose (e.g., full-dose image slice 103). In some cases, the raw input image may be 2D image slices. A deep learning (DL) model such as a U-net encoder-Decoder 111 model may be used to predict an inference result 112. While the DL model 111 may be a 2D model that is trained to generate an enhanced image within each slice, it may produce inconsistent image enhancement across slices such as streaking artifacts in image reformats. For instance, when the inference result is reformatted 113 to generate a reformat image in the orthogonal direction 114, because the input 2D image 110 matches the scanning plane, the reformat image 114 may contain reformat artifacts such as streaking artifacts in the orthogonal directions.

Such reformat artifacts may be alleviated by adopting a multi-planar reformat (MPR) method 120 and using a 2.5D trained model 131. The MPR method may beneficially augment the input volumetric data in multiple orientations. As shown in FIG. 1, a selected number of input slices of the pre-contrast image 101 and full-dose images 103 may be stacked channel-wise to create a 2.5D volumetric input image. The number of input slices for forming the 2.5D volumetric input image can be any number such as at least two, three, four, five, six, seven, eight, nine, ten slices may be stacked. In some cases, the number of input slices may be determined based on the physiologically or biochemically important structures in regions of interest such as microstructures where a volumetric image without artifacts are highly desired. For instance, the number of input slices may be selected such that microstructure (e.g., blood vessels or tumors) may be mostly contained in the input 2.5D volumetric image. Alternatively or additionally, the number of slices may be determined based on empirical data or selected by a user. In some cases, the number of slices may be optimized according the computational power and/or memory storage of the computing system.

Next, the input 2.5D volumetric image may be reformatted into multiple axes such as principal axes (e.g., sagittal, coronal, and axial) to generate multiple reformatted volumetric images 121. The multiple orientations for reformatting the 2.5D volumetric images may be in any suitable directions that need not be aligned to the principal axes. Additionally, the number of orientations for reformatting the volumetric images can be any number greater than one, two, three, four, five and the like so long as at least one of the multiple reformatted volumetric images is along an orientation that is oblique to or orthogonal to the scanning plane.

At inference stage, each of the multiple reformatted volumetric images may be rotated by a series of angles to produce a plurality of rotated reformat volumetric images 122 thereby further augmenting the input data. For example, each of the three reformatted volumetric images 121 (e.g., sagittal, coronal, and axial) may be rotated by five equispaced angles between 0-90° resulting in 15 volumetric images 122. It should be noted that the angle step and the angle range can be in any suitable range. For example, the angle step may not be a constant and the number of rotational angles can vary based on different applications, cases, or deployment scenarios. In another example, the volumetric images can be rotated across any angle range that is greater than, smaller than or partially overlapping with 0-90°. The effect of the number of the rotational angles on the predicted MPR images are described later herein.

The plurality of rotated volumetric 2.5D images 122 may then be fed to the 2.5D trained model 131 for inference. The output of the 2.5D trained model includes a plurality of contrast-enhanced 2.5 D volumetric images. In some cases, the final inference result 132, which is referred to as the “MPR reconstruction”, may be an average of the plurality of contrast-enhanced 2.5 D volumetric images after rotating back to the original acquisition/scanning plane. For instance, the 15 enhanced 2.5 D volumetric images may be rotated back to be aligned to the scanning plane and the mean of such volumetric images is the MPR reconstruction or the final inference result 132. The plurality of predicted 2.5 D volumetric images may be rotated to be aligned to the original scanning plane or the same orientation such that an average of the plurality of 2.5D volumetric images may be computed. The plurality of enhanced 2.5D volumetric images may be rotated to be aligned to the same direction that may or may not be in the original scanning plane. The MPR reconstruction method beneficially allows to add a 3D context to the network while benefitting from the performance gains of 2D processing.

As illustrated in FIG. 1, when the MPR reconstruction image 132 is reformatted 133 into a plane orthogonal to the original acquisition plane, the reformat image 135 does not present streaking artifacts. The quality of the predicted MPR reconstruction image may be quantified by quantitative image quality metrics such as peak signal to noise ratio (PSNR), and structural similarity (SSIM).

### Data Collection

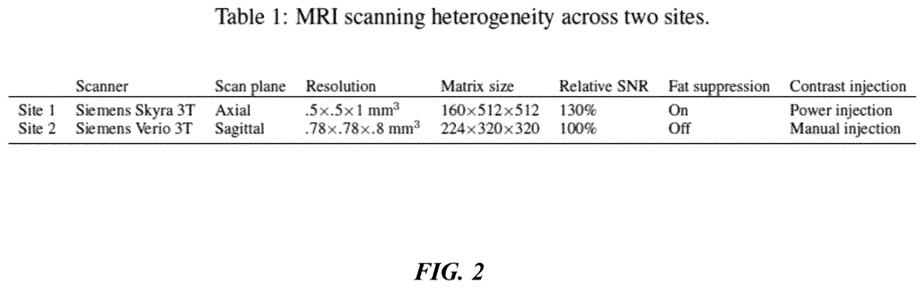

In an example, under IRB approval and patient consent, the scanning protocol was implemented in two sites. FIG. 2 shows the example of data collected from the two sites. 24 patients (16 training, 8 testing) were recruited from Site 1 and 28 (23 training, 5 testing) from Site 2. Differences between scanner hardware and protocol are highlighted in Table 1. In particular, the two sites used different scanner hardware, and had great variability in scanning protocol. Notably, Site 1 used power injection to administer GBCA, while Site 2 used manual injection, leading to differences in enhancement time and strength.

As an example of collecting data for training the model, multiple scans with reduced dose level as well as a full-dose scan may be performed. The multiple scans with reduced dose level may include, for example, a low-dose (e.g., 10%) contrast-enhanced MRI and a pre-contrast (e.g., zero contrast) may be performed. For instance, for each participant, two 3D T₁-weighted images were obtained: pre-contrast and post-10% dose contrast (0.01 mmol/kg). For training and clinical validation, the remaining 90% of the standard contrast dose (full-dose equivalent, 100%-dose) was administrated and a third 3D T₁-weighted image (100%-dose) was obtained. Signal normalization is performed to remove systematic differences (e.g., transmit and receive gains) that may have caused signal intensity changes between different acquisitions across different scanner platforms and hospital sites. Then, nonlinear affine co-registration between pre-dose, 10%-dose, and 100%-dose images are performed. The DL model used a U-Net encoder-decoder architecture, with the underlying assumption that the contrast-related signal between pre-contrast and low-dose contrast-enhanced images was nonlinearly scaled to the full-dose contrast images. Additionally, images from other contrasts such as T₂ and T₂-FLAIR can be included as part of the input to improve the model prediction.

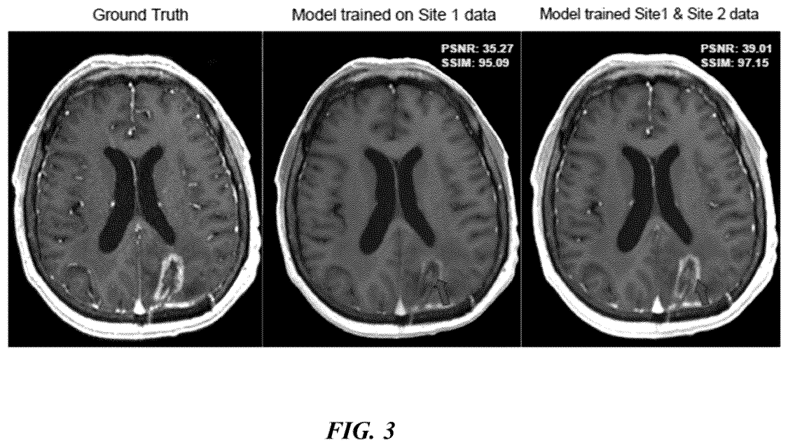

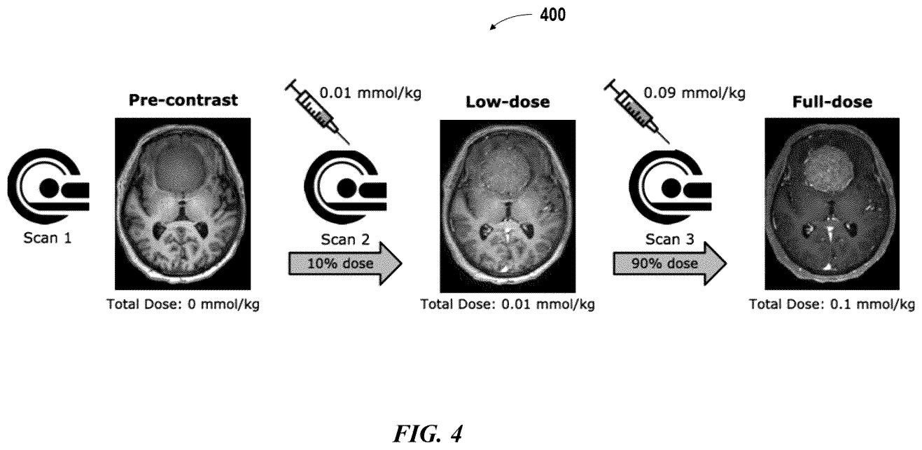

FIG. 4 shows an example of a scan procedure or scanning protocol 400 utilized for collecting data for the studies or experiments shown in FIGS. 2 and 3. In the illustrated scan protocol, each patient underwent three scans in a single imaging session. Scan 1 was pre-contrast 3D T₁-weighted MRI, followed by Scan 2 with 10% of the standard dose of 0.1 mmol/kg. Images from Scan 1 and 2 were used as input to the DL network. Ground truth images were obtained from Scan 3, after administering the remaining 90% of the contrast dose (i.e., full dose).

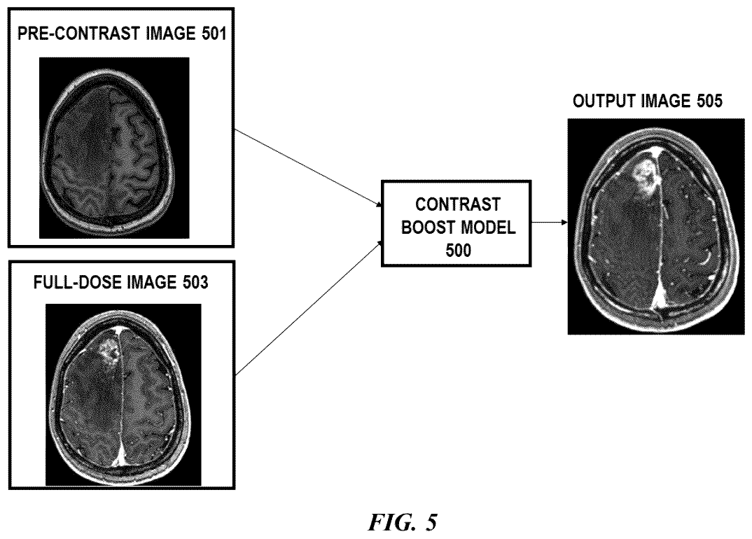

During inference, after deployment of the provided systems, one scan without contrast agent (e.g., similar to scan 1), and a scan with full contrast dose (e.g., similar to scan 3) or standard dosage of contrast agent may be performed. Such input image data may then be processed by the trained model to output a predicted MPR reconstructed image with enhanced contrast. FIG. 5 schematically shows an example of processing the input images including pre-contrast image 501 and full-dose image 503 with the trained contrast boost model 500, and output the contrast enhanced image 505. The pre-contrast images 501 is acquired without administering contrast agent and the full-dose images 503 are acquires with administering contrast agent at full-dose level or standard level. The contrast enhanced image 505 may have an image quality higher than both the full-dose image 504 and the pre-contrast image 501. For instance, the quality of the synthesized contrast enhanced image 505 may be same as an image acquired with a contrast agent dose level higher than a full-dose/standard dose level.

In some embodiments, both the pre-contrast image and full-dose image may be processed such as by applying the MPR method as described in FIG. 1. For instance, the pre-contrast image and full-dose image may be reformatted into multiple orientations to generate one or more reformatted input images and these reformatted input images may be fed to the trained contrast boost model 500 to output a predicted image (MPR reconstruction image) with improved quality. Alternatively, the MPR method may be applied to the full-dose image only such that the reformatted full-dose images along with the pre-contrast image may be fed to the trained contrast boost model 500 to output a predicted image with improved quality. In another example, the MPR method may be applied to the pre-contrast image only such that the reformatted pre-contrast images along with the full-dose image may be fed to the trained contrast boost model 500 to output a predicted image with improved quality.

### Inter-Site Generalizability

The conventional model may be limited by evaluating patients from a single site with identical scanning protocol. In real clinical settings, each site may tailor its protocol based on the capabilities of the scanner hardware and standard procedures. For example, a model trained on Site 2 may perform poorly on cases from Site 1 (FIG. 2, middle).

The provided DL model may have improved generalizability. The DL model may be trained with a proprietary training pipeline. For example, the training pipeline may comprise first scaling each image to a nominal resolution of 1 mm³ and in-plane matrix size of 256×256, followed by applying the MPR processing. As the DL model is fully convolutional, inference can be run at the native resolution of the acquisition without resampling.

Based on the qualitative and quantitative results, the addition of MPR processing, resolution re-sampling, and inter-site training beneficially provides improvement in model robustness and generalizability. In optional embodiments, the model may be a full 3D model. For instance, the model may be a 3D patch-based model that may alleviate both MPR processing, and memory usage. The provided training methods and model framework may be applied to different sites with different scanner platforms, and/or across different MRI vendors.

### Network Architecture and Processes

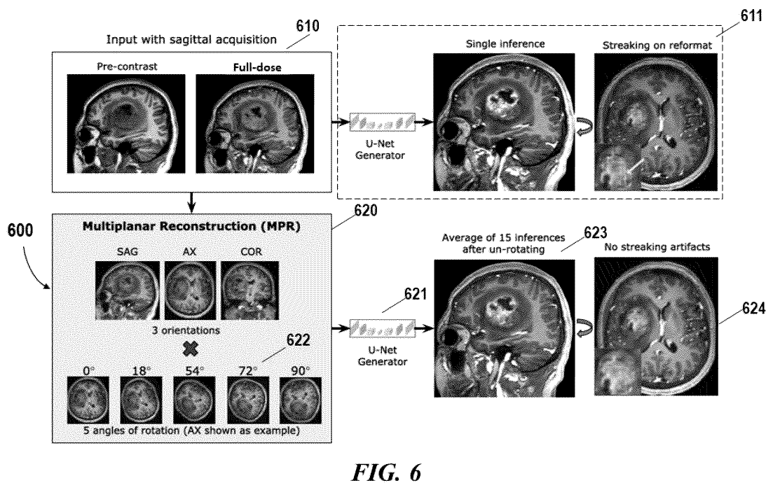

FIG. 6 schematically illustrates another example of an MPR reconstructed image 624 that have improved quality compared to the MRI image predicted using the conventional method 611. The workflow 600 for processing and reconstructing MRI volumetric image data 623 and the reformat MPR reconstructed image 624 can be the same as those as described in FIG. 1. For example, the input image 610 may include a plurality of 2D image slices that are acquired without contrast agent (e.g., pre-contrast image slice) and/or with full contrast dose (e.g., full-dose image slice). The input images may be acquired in a scanning plane (e.g., axial) or along a scanning orientation. A selected number of the image slices are stacked to form a 2.5D volumetric input image which is further processed using the multiplanar reconstruction (MPR) method 620 as described above.

For example, the input 2.5D volumetric image may be reformatted into multiple axes such as principal axes (e.g., sagittal, coronal, and axial) to generate multiple reformatted volumetric images (e.g., SAG, AX, COR). It should be noted that the 2.5D volumetric image can be reformatted into any orientations that may or may not be aligned with the principal axes.

Each of the multiple reformatted volumetric images may be rotated by a series of angles to produce a plurality of rotated reformat images. For example, each of the three reformatted volumetric images (e.g., sagittal, coronal, and axial) may be rotated by five angles between 0-90° resulting in 15 rotated reformat volumetric images. The multiple reformatted volumetric images (e.g., sagittal, coronal, and axial) may or may not be rotated at the same angle or rotated into the same number of orientations.

The plurality of rotated volumetric images 622 may then be processed by the trained model 621 to produce a plurality of enhanced volumetric images. In some cases, the MPR reconstruction image 623 or the inference result image is the average of the plurality of inference volumes after rotating back to the original plane of acquisition. The MPR reconstruction image when is reformatted to be viewed at a selected orientation (e.g., orthogonal/oblique to the scanning plane), the reformat image 624 may not contain streaking artifacts compared to the reformat image obtained using the single inference method 611 and/or the single inference model.

### Network Architecture and Data Processing

Using the multiplanar reconstruction (MPR) technique, the deep learning model (e.g., contrast boost model 500) may be trained with volumetric images (e.g., augmented 2.5D images) such as from the multiple orientations (e.g., three principal axes). The contrast boost model may be a trained deep learning model for enhancing the quality of volumetric MRI images. The MIR images may be acquired using full contrast dose. In some embodiments, the model may include an artificial neural network that can employ any type of neural network model, such as a feedforward neural network, radial basis function network, recurrent neural network, convolutional neural network, deep residual learning network and the like. In some embodiments, the machine learning algorithm may comprise a deep learning algorithm such as convolutional neural network (CNN). Examples of machine learning algorithms may include a support vector machine (SVM), a naïve Bayes classification, a random forest, a deep learning model such as neural network, or other supervised learning algorithm or unsupervised learning algorithm. The model network may be a deep learning network such as CNN that may comprise multiple layers. For example, the CNN model may comprise at least an input layer, a number of hidden layers and an output layer. A CNN model may comprise any total number of layers, and any number of hidden layers. The simplest architecture of a neural network starts with an input layer followed by a sequence of intermediate or hidden layers, and ends with output layer. The hidden or intermediate layers may act as learnable feature extractors, while the output layer in this example provides 2.5D volumetric images with enhanced quality (e.g., enhanced contrast). Each layer of the neural network may comprise a number of neurons (or nodes). A neuron receives input that comes either directly from the input data (e.g., low quality image data, image data acquired with reduced contrast dose, etc.) or the output of other neurons, and performs a specific operation, e.g., summation. In some cases, a connection from an input to a neuron is associated with a weight (or weighting factor). In some cases, the neuron may sum up the products of all pairs of inputs and their associated weights. In some cases, the weighted sum is offset with a bias. In some cases, the output of a neuron may be gated using a threshold or activation function. The activation function may be linear or non-linear. The activation function may be, for example, a rectified linear unit (ReLU) activation function or other functions such as saturating hyperbolic tangent, identity, binary step, logistic, arcTan, softsign, parameteric rectified linear unit, exponential linear unit, softPlus, bent identity, softExponential, Sinusoid, Sinc, Gaussian, sigmoid functions, or any combination thereof.

In some embodiments, the network may be an encoder-decoder network or a U-net encoder-decoder network. A U-net is an auto-encoder in which the outputs from the encoder-half of the network are concatenated with the mirrored counterparts in the decoder-half of the network. The U-net may replace pooling operations by upsampling operators thereby increasing the resolution of the output.

In some embodiments, the contrast boost model for enhancing the volumetric image quality may be trained using supervised learning. For example, to train the deep learning network, pairs of pre-contrast and low-dose images as input and the full-dose image as the ground truth from multiple subjects, scanners, clinical sites or databases may be provided as training dataset.

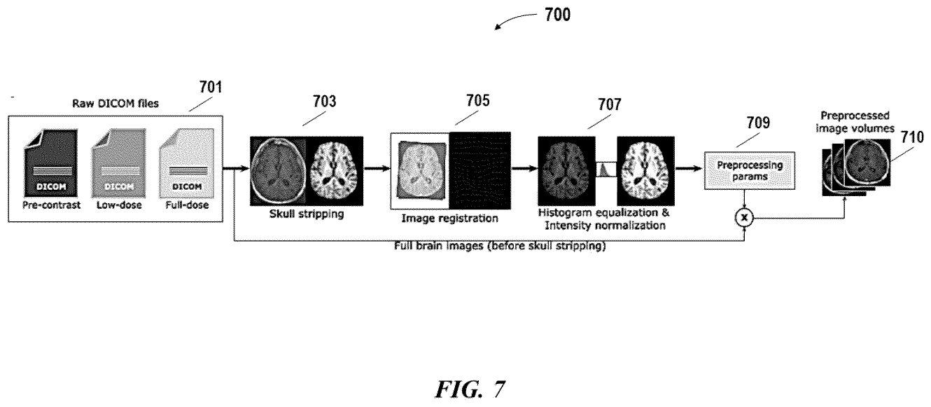

In some cases, the input datasets may be pre-processed prior to training or inference. FIG. 7 shows an example of a pre-processing method 700, in accordance with some embodiments herein. As shown in the example, the input data including the raw pre-contrast, low-dose, and full-dose image (i.e., ground truth) may be sequentially preprocessed to generate preprocessed image data 710. The raw image data may be received from a standard clinical workflow, as a DICOM-based software application or other imaging software applications. As an example, the input data 701 may be acquired using a scan protocol as described in FIG. 4. For instance, three scans including a first scan with zero contrast dose, a second scan with a reduced dose level and a third scan with full dose may be operated. The reduced dose image data used for training the model, however, can include images acquired at various reduced dose level such as no more than 1%, 5%, 10%, 15%, 20%, any number higher than 20% or lower than 1%, or any number in-between. For example, the input data may include image data acquired from two scans including a full dose scan as ground truth data and a paired scan at a reduced level (e.g., zero dose or any level as described above). Alternatively, the input data may be acquired using more than three scans with multiple scans at different levels of contrast dose. Additionally, the input data may comprise augmented datasets obtained from simulation. For instance, image data from clinical database may be used to generate low quality image data mimicking the image data acquired with reduced contrast dose. In an example, artifacts may be added to raw image data to mimic image data reconstructed from images acquired with reduced contrast dose.

In the illustrated example, pre-processing algorithm such as skull-stripping 703 may be performed to isolate the brain image from cranial or non-brain tissues by eliminating signals from extra-cranial and non-brain tissues using the DL-based library. Based on the tissues, organs and use application, other suitable preprocessing algorithms may be adopted to improve the processing speed and accuracy of diagnosis. In some cases, to account for patient movement between the three scans, the low-dose and full-dose images may be co-registered to the pre-contrast image 705. In some cases, given that the transmit and receive gains may vary for different acquisitions, signal normalization may be performed through histogram equalization 707. Relative intensity scaling may be performed between the pre-contrast, low-dose, and full-dose for intra-scan image normalization. As the multi-institutional dataset include images with different voxel and matrix sizes, the 3D volume may be interpolated to an isotropic resolution of 0.5 mm³ and wherever applicable, zero-padded images at each slice to a dimension of 512×512. The image data may have sufficiently high resolution to enable the DL network to learn small enhancing structures, such as lesions and metastases. In some cases, scaling and registration parameters may be estimated on the skull-stripped images and then applied to the original images 709. The preprocessing parameters estimated from the skull-stripped brain may be applied to the original images to obtain the preprocessed image volumes 710.

Next, the preprocessed image data 710 is used to train an encoder-decoder network to reconstruct the contrast-enhanced image. The network may be trained with an assumption that the contrast signal in the full-dose is a non-linearly scaled version of the noisy contrast uptake between the low-dose and the pre-contrast images. The model may not explicitly require the difference image between low-dose and pre-contrast.

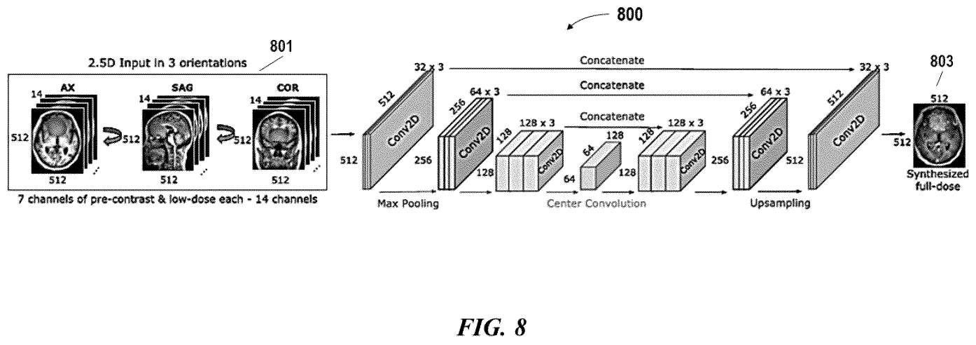

FIG. 8 shows an example of a U-Net style encoder-decoder network architecture 800, in accordance with some embodiments herein. In the illustrated example, each encoder block has three 2D convolution layers (3×3) with ReLU followed by a maxpool (2×2) to downsample the feature space by a factor of two. The decoder blocks have a similar structure with maxpool replaced with upsample layers. To restore spatial information lost during downsampling and prevent resolution loss, decoder layers are concatenated with features of the corresponding encoder layer using skip connections. The network may be trained with a combination of L1 (mean absolute error) and structural similarity index (SSIM) losses. Such U-Net style encoder-decoder network architecture may be capable of producing a linear 10× scaling of the contrast uptake between low-dose and zero-dose, without picking up noise along with the enhancement signal.

As shown in FIG. 8, the input data to the network may be a plurality of augmented volumetric images generated using the MPR method as described above. In the example, seven slices each of pre-contrast and low-dose images are stacked channel-wise to create a 14-channel input volumetric data for training the model to predict the central full-dose slices 803.

### Enhancement and Weighted L1 Loss

In some situations, even after signal normalization and scaling is applied, the difference between the low-dose and pre-contrast images may have enhancement-like noise perturbations which may mislead training of the network. To make the network pay more attention to the actual enhancement regions, the L1 loss may be weighted with an enhancement mask. The mask is continuous in nature and is computed from the skull-stripped difference between low-dose and pre-contrast images, normalized between 0 and 1. The enhancement mask can be considered as a normalized smooth version of the contrast uptake.

### Perceptual and Adversarial Losses

It is desirable to train the network to focus on the structural information in the areas of enhancement as well as high frequency and texture details which are crucial for making confident diagnostic decisions. A simple combination of L1 and structural similarity index (SSIM) losses may tend to suppress high-frequency signal information and the obtained results may have a smoother appearance, which is perceived as a loss of image resolution. To address this issue, a perceptual loss from a convolutional network (e.g., VGG-19 network consisting of 19 layers including 6 convolution layers, 3 Fully connected layer, 5 MaxPool layers and 1 SoftMax layer which is pre-trained on ImageNet dataset) is employed. The perceptual loss is effective in style-transfer and super-resolution tasks. For example, the perceptual loss can be computed from the third convolution layer of the third block (e.g., block3 conv3) of a VGG-19 network, by taking the mean squared error (MSE) of the layer activations on the ground truth and prediction.

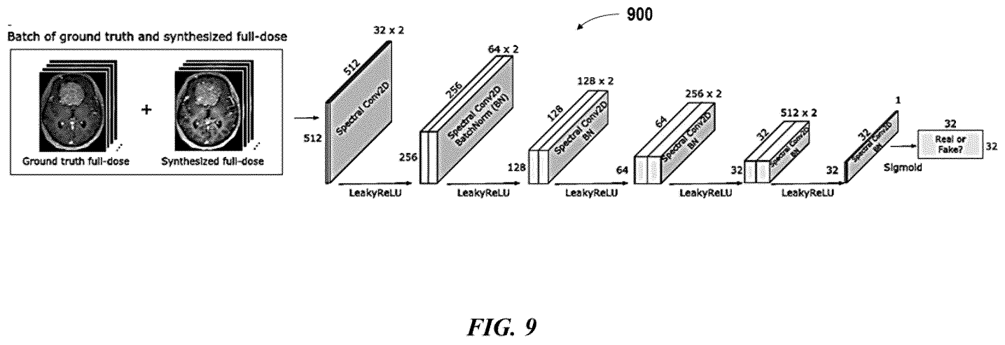

In some cases, to further improve the overall perceptual quality, an adversarial loss is introduced through a discriminator, trained in parallel to the encoder-decoder network, to predict whether the generated image is real or fake. FIG. 9 shows an example of the discriminator 900, in accordance with some embodiments herein. The discriminator 900 has a series of spectral normalized convolution layers with Leaky ReLU activations and predicts a 32×32 patch. The discriminator 900 is trained to discriminate between the ground truth full-dose image and the synthesized full-dose image. Unlike a conventional discriminator, which predicts a binary value (e.g., 0 for fake and 1 for real), the “patch discriminator” 900 predicts a matrix of probabilities which helps in the stability of the training process and faster convergence. The spectral normalized convolution layer employs a weight normalization technique to further stabilize discriminator training. The patch discriminator, as shown in FIG. 9, can be trained with MSE loss, and Gaussian noise may be added to the inputs for smooth convergence.

The function for configuring the network model can be formulated as below:

G*=argmin_{G}[λ_{GAN}L_{GAN}(G)+λ_{L1}L_{L1}(M_{enh·}G)+λ_{SSIM}L_{SSIM}(G)+λ_{VGG}L_{VGG}(G)]

where Mₑₙₕ is the enhancement mask and the adversarial loss L_{GAN} can be written as L_{GAN}=max_{D}L_{GAN}(G, D), where G is the U-Net generator and D is the patch-discriminator. The loss weights λ_{L}, λ_{SSIM}, λ_{VGG} and λ_{GAN} can be determined empirically. With the abovementioned processes and methods, a single model is trained to make accurate predictions on images from various institutions and scanners. The model (i.e., contrast boost model) is trained to enhance the contrast. The output of the model is synthesized contrast enhanced images. The contrast enhanced images have an image quality (e.g., noise, image contrast, etc.) that is similar to the image acquired with increased contrast agent. The quality of the synthesized contrast enhanced image may be improved over the quality of the input image.

### Example

FIG. 3 shows an example of analytic results of a study to evaluate the generalizability and accuracy of the contrast boost model. In the illustrated example, the results show comparison of ground-truth (left), original model (middle), and proposed model (right) inference result on a test case from Site 1 (red arrow shows lesion conspicuity). The conventional model was trained on data from Site 2 only. This example is consistent with the MRI scanning data illustrated in FIG. 2. The provided model was trained on data from both sites, and used MPR processing and resolution resampling. In this study, the result qualitatively shows the effect of MPR processing on one example from the test set. By averaging the result of many MPR reconstructions, streaking artifacts that manifest as false enhancement are suppressed. As shown in FIG. 3, one slice of a ground-truth contrast-enhanced image (left) is compared to the inference results from the model trained on Site 2 (middle) and the model trained on Sites 1 and 2 simultaneously (right). By accounting for differences in resolution and other protocol deviations, the provided model demonstrates qualitative improvement in generalizability. Quantitative image quality metrics such as peak signal to noise ratio (PSNR), and structural similarity (SSIM) were calculated for all the conventional model and the presented model. The average PSNR and SSIM on the test set for the conventional and presented model was 32.81 dB (38.12 dB) and 0.872 (0.951), respectively. Better image quality may be achieved using the methods and systems in the present disclosure.

In the study as illustrated in FIG. 3, a deep learning (DL) framework as described elsewhere herein is applied for low-dose (e.g., 10%) contrast-enhanced MRI. For each participant, two 3D T₁-weighted images were obtained: pre-contrast and post-10% dose contrast (0.01 mmol/kg). For training and clinical validation, the remaining 90% of the standard contrast dose (full-dose equivalent, 100%-dose) was administrated and a third 3D T₁-weighted image (100%-dose) was obtained. Signal normalization was performed to remove systematic differences (e.g., transmit and receive gains) that may have caused signal intensity changes between different acquisitions across different scanner platforms and hospital sites. Then, nonlinear affine co-registration between pre-dose, 10%-dose, and 100%-dose images were performed. The DL model used a U-Net encoder-decoder architecture, with the underlying assumption that the contrast-related signal between pre-contrast and low-dose contrast-enhanced images was nonlinearly scaled to the full-dose contrast images. Images from other contrasts such as T₂ and T₂-FLAIR can be included as part of the input to improve the model prediction.

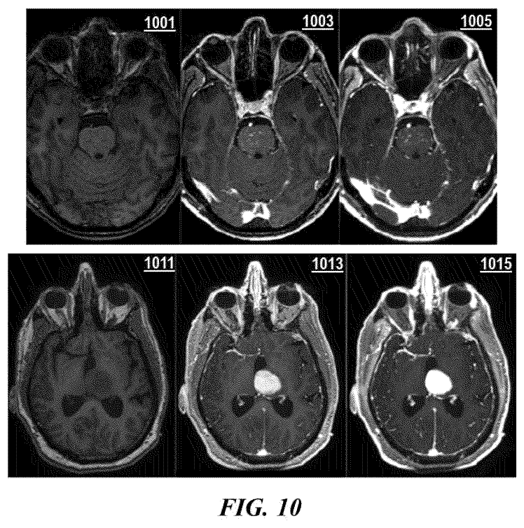

FIG. 10 shows examples of contrast enhanced images outputted by the contrast boost model. The input to the model may comprise pre-contrast image 1001, 1011 (e.g., images acquired without contrast agent), full-dose images 1003, 1013 (e.g., images acquired with contrast agent at full-dose complying with a standard protocol). The output images 1005, 1015 as shown in the example have image quality improved over the full-dose images. In particular, the output images have an image quality similar to the quality of the image acquired with increased contrast agent dose (i.e., dose level higher than full-dose). This beneficially allowing for enhancing image quality without requiring extra contrast agent. The pre-contrast agent and full-dose image acquisitions can be made in a single imaging session.

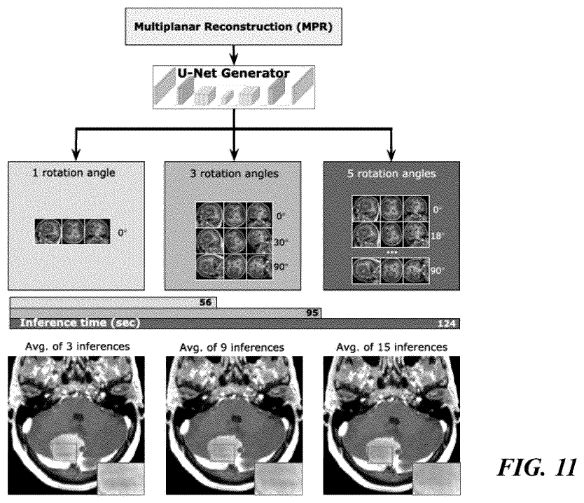

FIG. 11 shows examples of different number of rotations and the corresponding effect on the quality of the image and the performance. The effect of the number of rotation angles in MPR as shown in FIG. 11 provides that greater number of angles may reduce the horizontal streaks inside the tumor (better quality), while it may also increase the inference time. When deploy a trained model to a physical site, the number of rotations and different angles may be determined based on the desired image quality and deployment environment (e.g., computational power, memory storage, etc.).

### Selective Processing Path

In an aspect of the present disclosure, the contrast boost model that is trained to boost contrast (i.e., predicting contrast-enhanced images) can be utilized in combination with other models that are trained to denoise the image, and/or synthesize super-resolution image. The methods and systems of the present disclosure beneficially provide various combination of the models and mechanism. In some cases, the input images may be processed by a first model trained to predict contrast-enhanced image, a second model trained to denoise the image, and a third model to improve the resolution of the image (e.g., super resolution image). The images may be processed in a selected processing path that comprises a combination of the above models in a selected or pre-determined order. In some cases, a processing path may comprise repeatedly applying one or more of the models. The processing path may comprise multi-contrast branched architecture which is described later herein.

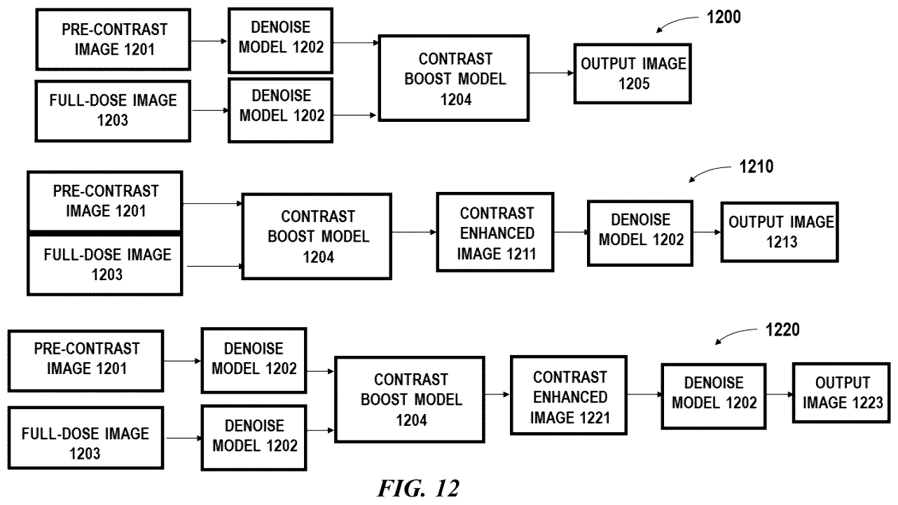

FIG. 12 shows multiple exemplary processing paths comprising various combinations of a contrast boost model 1204 and a denoise model 1202. The processed illustrated in FIG. 12 may be in an inference stage. A processing path 1200, 1210, 1220 may comprise a contrast boost model 1204 and a denoise model 1201 organized/arranged in predetermined order to process an input image. The input image may comprise a pre-contrast image 1201 and a full-dose image 1203. The pre-contrast image 1201 may be acquired without administering contrast agent and the full-dose image 1203 may be acquired with contrast agent at full-dose level according to a standard protocol. The acquisition method can be the same as those described above. The contrast boost model 1204 can be the same as the DL model as described above. For example, the input images may be processed by the multiplanar reconstruction methods and the contrast boost model may be a 2.5D deep learning model or 3D model as described above.

In some embodiments, the denoise model 1202 may be a deep learning model that is trained to improve quality image. The output image of the denoise model 1202 may have greater SNR, higher resolution, or less aliasing compared with the input image to the denoise model.

The denoise model 1202 may be a deep learning model trained using training datasets comprising at least a low-quality image and a high-quality image. In some cases, the low-quality image is generated by applying one or more filters or adding synthetic noise to the high-quality image to create noise or undersampling artifacts. In some cases, the denoise model 1202 may be trained using image patches that comprise a portion of at least a low quality image and a high quality image. In some cases, one or more patches may be selected from a set of patches and used for training the model. In some instances, one or more patches corresponding to the same coordinates may be selected from a pair of images. Alternatively, a pair of patches may not correspond to the same coordinates. The selected pair of patches may then be used for training. In some cases, patches from the pair of images with similarity above a pre-determined threshold are selected. One or more pairs of patches may be selected using any suitable metrics quantifying image similarity. For instance, one or more pairs of patches may be selected by calculating a structural similarity index, peak signal-to-noise ratio (PSNR), mean squared error (MSE), absolute error, other metrics or any combination of the above. In some cases, the similarity comparison may be performed using sliding window over the image.

In some cases, the training process of the denoise model 1202 may employ a residual learning method. In some instances, the residual learning framework may be used for evaluating a trained model. In some instances, the residual learning framework with skip connections may generate estimated ground-truth images from the lower quality images such as complex-valued aliased ones, with refinement to ensure it is consistent with measurement (data consistency). The lower quality input image can be simply obtained via inverse Fourier Transform (FT) of undersampled data. In some cases, what the model learns is the residual of the difference between the raw image data and ground-truth image data, which is sparser and less complex to approximate using the network structure. The method may use by-pass connections to enable the residual learning. In some cases, a residual network may be used and the direct model output may be the estimated residual/error between low-quality and high quality images. In other word, the function to be learned by the deep learning framework is a residual function which in some situations may be easy to optimize. The higher quality image can be recovered by adding the low quality image to the residual. The model architecture and training method for the denoise model 1202 can include those described in U.S. Ser. No. 11/182,878 entitled “Systems and methods for improving magnetic resonance imaging using deep learning” which is incorporated by reference herein in its entirety.

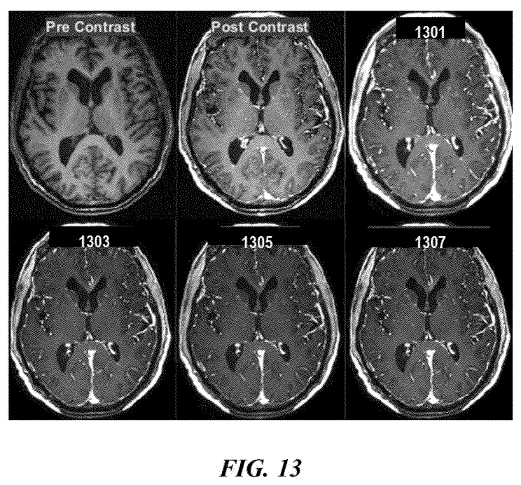

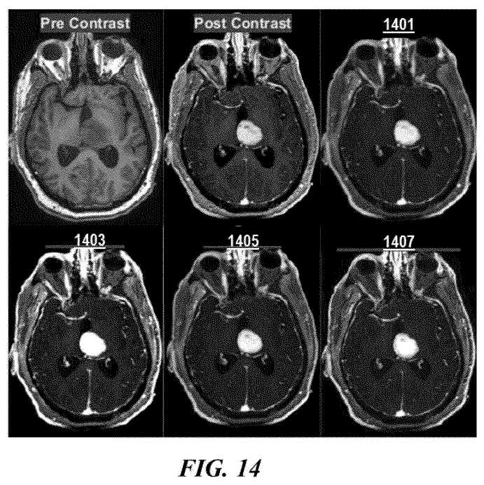

In a first exemplary processing path 1200, the pre-contrast image 1201 and full-dose image 1203 may be processed by the denoise model 1202 respectively, the output of the denoise model 1202 may then be processed by the contrast boost model 1204 to generate the final output image 1205. FIG. 13 and FIG. 14 show examples of the output image 1303, 1403 generated by the processing path 1200. A synthesized image 1301, 1401 generated with the contrast boost model only (without applying the denoise model) is compared against the output image generated by the different processing paths 1200, 1210, 1220.

In a second exemplary processing path 1210, the pre-contrast image 1201 and full-dose image 1203 may be processed by the contrast boost model 1204 first to generate contrast enhanced image 1211. As described above, the contrast enhanced image may mimic an image acquired with increased dose of contrast agent compared to the full-dose level. The contrast-enhanced image 1211 may then be processed by the denoise model 1202 to generate the final output image 1213. FIG. 13 and FIG. 14 show examples of the output image 1305, 1405 generated by the processing path 1210.

In a third exemplary processing path 1220, the pre-contrast image 1201 and full-dose image 1203 may be processed by the denoise model 1202 respectively, the output of the denoise model 1202 may then be processed by the contrast boost model 1204 to generate contrast enhanced image 1221. The contrast enhanced image 1221 may be processed by the denoise model 1202 again to output the final output image 1223. FIG. 13 and FIG. 14 show examples of the output image 1307, 1407 generated by the processing path 1220.

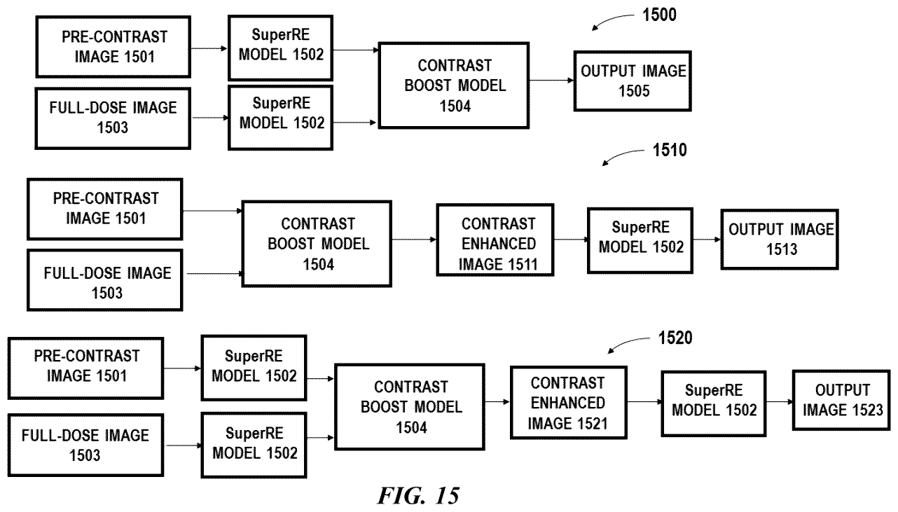

FIG. 15 shows multiple exemplary processing paths comprising various combinations of a contrast boost model 1504 and a super-resolution model 1502. A processing path 1500, 1510, 1520 may comprise a contrast boost model 1504 and a denoise model 1501 organized in predetermined order to process an input image. The input image may comprise a pre-contrast image 1501 and a full-dose image 1503. The pre-contrast image 1501 may be acquired without administering contrast agent and the full-dose image 1503 may be acquired with contrast agent at full-dose level according to a standard protocol. The acquisition method can be the same as those described above. The contrast boost model 1504 can be the same as the DL model as described elsewhere herein. For example, the input images may be processed by the multiplanar reconstruction methods and the contrast boost model may be a 2.5D deep learning model or 3D model as described above.

In some embodiments, the super resolution model 1502 may be a deep learning model that is trained to improve quality image. The output image of the super resolution model 1502 may have greater SNR, higher resolution, or less aliasing compared with the input image to the denoise model. In some cases, the super resolution model 1502 may be trained to predict ultra-high resolution image.

In some embodiments, the super resolution model 1502 may be trained based on ground truth data that include high SNR and high resolution images acquired using a longer scan. In some embodiments, the super resolution model 1502 may be a highly nonlinear mapping from low image quality to high image quality images. In some embodiments, the super resolution model 1502 may be based on relativistic generative adversarial network (GAN) with gradient guidance. For instance, the model may use gradient maps as side information to recover more perceptual-pleasant details thereby increasing resolution and fine details. In some cases, the super resolution model may generate predicted gradient maps of high-resolution images using additional gradient branch to assist a super resolution (SR) reconstruction task. For example, the super resolution model may avoid fake details generated by GAN by using L₁ loss and perceptual loss. By adjusting the weights of adversarial loss, perceptual loss, pixel-wise loss and gradient loss, as well as applying the guidance of gradient branch, the framework can directly learn the mapping from data pairs (e.g., clinical data pairs) and reconstruct reliable super resolution images. In some cases, the model may comprise a main super resolution (SR) branch configured to take low resolution image as inputs and generate super resolution images, and a gradient branch configured to take the gradient maps of the low resolution image as input and guide the main branch using gradient maps of the super resolution images predicted by the main branch. The model architecture and training method for the super resolution model can include those described in U.S. Ser. No. 10/096,109 entitled “Quality of medical images using multi-contrast and deep learning” and PCT/CN2021/122318 entitled “ULTRA-HIGH RESOLUTION CT RECONSTRUCTION USING GRADIENT GUIDANCE” which are incorporated by reference herein in their entirety.

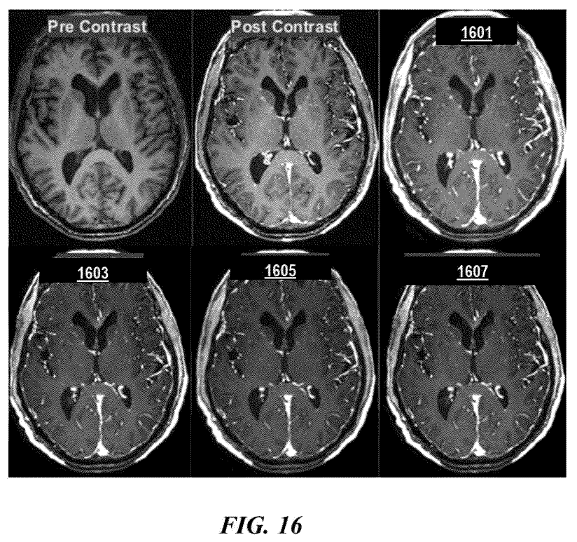

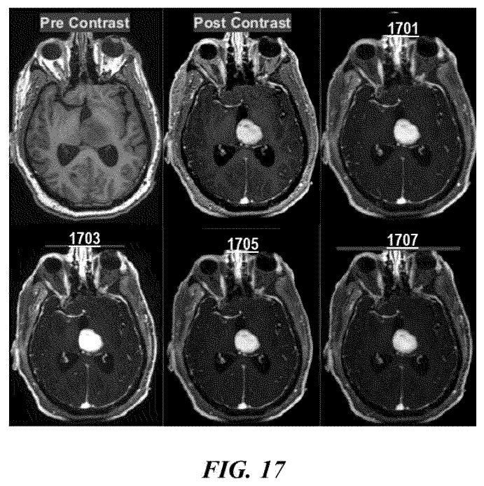

In a first exemplary processing path 1500, the pre-contrast image 1501 and full-dose image 1503 may be processed by the super resolution model 1502 respectively, the output of the super resolution model 1502 may then be processed by the contrast boost model 1504 to generate the final output image 1505. FIG. 16 and FIG. 17 show examples of the output image 1603, 1703 generated by the processing path 1500. A synthesized image 1601, 1701 generated with the contrast boost model only (without applying the super resolution model) is compared against the output image generated by the different processing paths 1500, 1510, 1520.

In a second exemplary processing path 1510, the pre-contrast image 1501 and full-dose image 1503 may be processed by the contrast boost model 1504 first to generate contrast enhanced image 1511. As described above, the contrast enhanced image may mimic an image acquired with increased dose of contrast agent compared to the full-dose level. The contrast-enhanced image 1511 may then be processed by the super resolution model 1502 to generate the final output image 1513. FIG. 16 and FIG. 17 show examples of the output image 1605, 1705 generated by the processing path 1510.

In a third exemplary processing path 1520, the pre-contrast image 1501 and full-dose image 1503 may be processed by the super resolution model 1502 respectively, the output of the super resolution model 1502 may then be processed by the contrast boost model 1504 to generate contrast enhanced image 1521. The contrast enhanced image 1521 may be then processed by the super resolution model 1502 again to output the final output image 1523. FIG. 16 and FIG. 17 show examples of the output image 1607, 1707 processed by the processing path 1520.

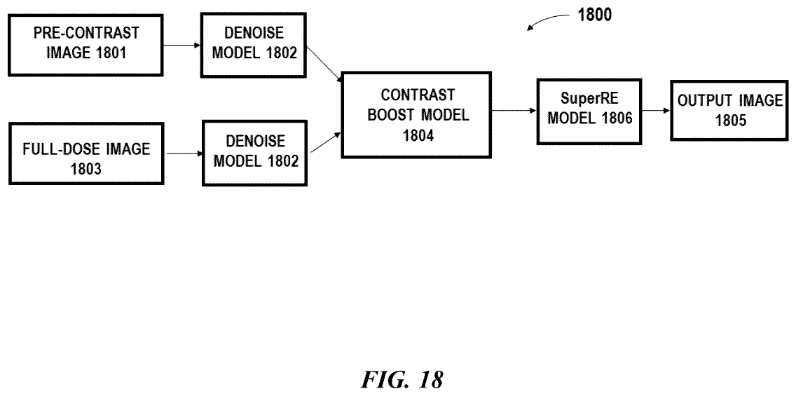

It should be noted that the processing path can include a combination of any of the above modes in a pre-determined order. For example, as shown in FIG. 18, the processing path 1800 may comprise a combination of the denoise model 1802, the contrast boost model 1804, and the super resolution model 1806. The pre-contrast image 1801 and the full-dose image 1803 may be processed by the processing path 1800 and generate a final output image 1805 with improved image quality.

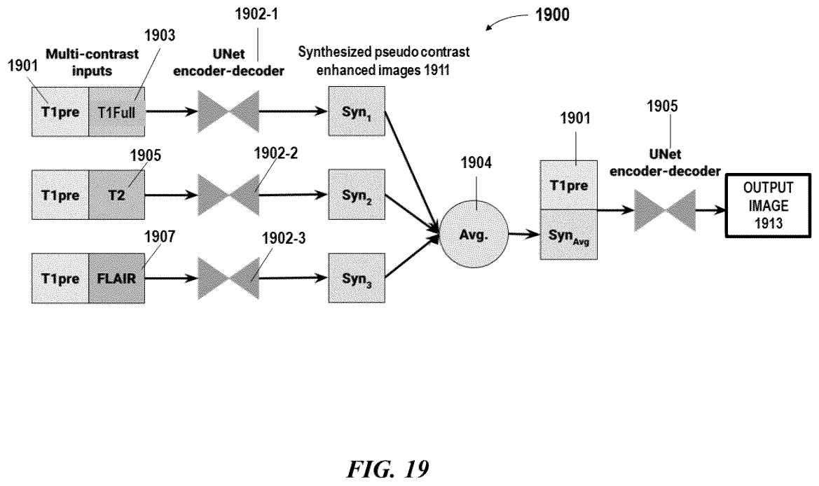

In some embodiments, the processing path may comprise a multi-contrast branched architecture. FIG. 19 shows an example of a processing path 1900 comprising a multi-contrast branched architecture. Each individual branch may comprise a contrast boost model 1902-1, 1902-2, 1902-3 to process different combinations of input images. The contrast boost model 1902-1, 1902-2, 1902-3 may be pre-trained with a full-dose contrast image as target. Unlike conventional methods or models squashing the multiple modalities as channels and supply the multiple-channel input to a single DL model, the multi-contrast branched architecture herein provides separate pathways for the individual contrasts encodings (e.g., T1, T2, FLAIR) each pathway comprises a respective DL model. Such multi-contrast branched architecture has improved performance compared to conventional method as the separate encoders are trained to learn the unique features offered by the different contrasts (i.e., multi-contrast inputs). For instance, the encoders of the contrast boost models 1902-1, 1902-2, 1902-3 in the separate paths may be trained to learn the features in the respective contrast-weighted images.

The input to the multi-contrast branched architecture may include different images acquired using different MRI imaging pulse sequences (e.g., contrast-weighted images such as T1-weighted (T1), T2-weighted (T2), proton density (PD) or Fluid Attenuation by Inversion Recovery (FLAIR), etc.). The input image to each branch or pathway may be different. For instance, different combinations of contrast images may be fed to different pathways. Different combinations may comprise different dose levels of contrast agent administered to a subject, different pulse sequences for acquiring the image or a combination of both.

The individual encoder-decoder pathways may combine the T1 pre-contrast 1901 with the respective contrasts T1 full-dose 1903, T2 1905, and FLAIR 1907 and output synthesized pseudo contrast enhanced images 1911. A plurality of synthesized pseudo contrast enhanced images 1911 generated by the multiple branches may be aggregated 1904. For example, the plurality of synthesized pseudo contrast enhanced images 1911 may be averaged 1904 and the averaged synthesized image may be combined with T1 pre-contrast image 1901 again and run through a final encoding pathway 1905 to generate the resulting image or final output image 1913. In some cases, the T2 1905 and FLAIR 1907 may be obtained before administering the contrast dose.

The learned contrast enhancement signals from the individual pathways may be boosted in the final encoder-decoder pathway to produce the final output image. The separate encoding pathways may be separately pre-trained with the respective combinations, to predict the contrast-enhanced images. For example, separate contrast boost models 1902-1, 1902-2, 1902-3 may be trained on T2 and FLAIR images to predict contrast enhanced images corresponding to the respective images.

In some embodiments, a processing path may be selected from a plurality of processing paths based on the quality of the input image, the use application (e.g., anomaly detection), user preference, the subject being imaged (e.g., organ, tissue), or any other conditions. Systems and methods herein may allow a user to customize or edit a processing path. In some cases, the provided system may automatically create a processing path based on a user input (e.g., user selected use application, tissue/organ being imaged, etc.) or based on simulated result. For instance, the system may generate simulated output results for different processing paths and may select a processing path based on a comparison of the performance (e.g., output image quality, processing time, etc.). Alternatively or additionally, a user may be presented with the simulated output results and permitted to select a processing path. In some cases, the simulated output results may be generated by processing a patch of the input images (e.g., a portion of the input image) thereby reducing the simulation time. In some cases, the system may determine whether the current input images are similar to images processed in a previous session, and may select a stored processing path based on the similarity. In some cases, once an optimal processing path is determined (e.g., a combination of denoise model-contrast boost model-denoise 1220), the optimal processing path along with characteristics of the project (e.g., organ, tissue being imaged, desired processing time, etc.) may be stored in a database for future application to a similar project.

### System Overview

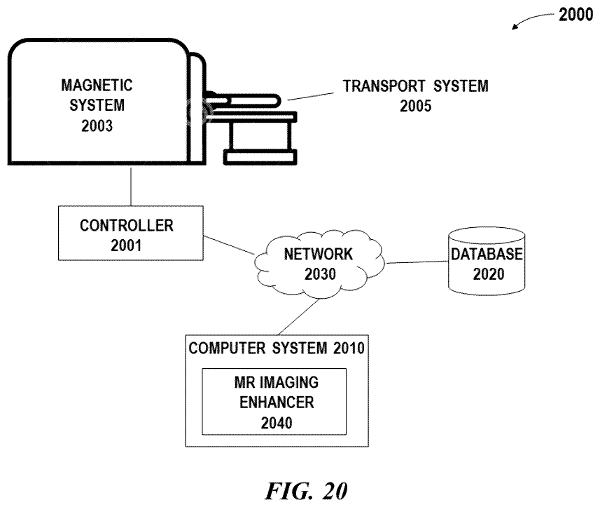

FIG. 20 schematically illustrates a magnetic resonance imaging (MRI) system 2000 in which an imaging enhancer 2040 of the presenting disclosure may be implemented. The MRI system 2000 may comprise a magnet system 2003, a patient transport table 2005 connected to the magnet system, and a controller 2001 operably coupled to the magnet system. In one example, a patient may lie on the patient transport table 2005 and the magnet system 2003 would pass around the patient. The controller 2001 may control magnetic fields and radio frequency (RF) signals provided by the magnet system 2003 and may receive signals from detectors in the magnet system 2003.

The MRI system 2000 may further comprise a computer system 2010 and one or more databases operably coupled to the controller 2001 over the network 2030. The computer system 2010 may be used for implementing the volumetric MR imaging enhancer 2040. The volumetric MR imaging enhancer 2040 may implement the contrast boost model, the different processing paths, the denoise model, the super resolution model, the multi-contrast branched processing path, and other methods described elsewhere herein. For example, the volumetric MR imaging enhancer may employ the MPR reconstruction method and various other training algorithms, and data processing methods, the processing path selection described herein. The computer system 2010 may be used for generating an imaging enhancer using training datasets. Although the illustrated diagram shows the controller and computer system as separate components, the controller and computer system can be integrated into a single component.

The computer system 2010 may comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, or a plurality of processors for parallel processing. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The MRI system 2000 may include one or more databases 2020 that may utilize any suitable database techniques. For instance, structured query language (SQL) or “NoSQL” database may be utilized for storing the reconstructed/reformat image data, raw collected data, training datasets, trained model (e.g., hyper parameters), weighting coefficients, rotation angles, rotation numbers, orientation for reformat reconstruction, processing paths, order or combination of different models, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present invention. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network 2030 may establish connections among the components in the MRI platform and a connection of the MRI system to external systems. The network 2030 may comprise any combination of local area and/or wide area networks using both wireless and/or wired communication systems. For example, the network 2030 may include the Internet, as well as mobile telephone networks. In one embodiment, the network 2030 uses standard communications technologies and/or protocols. Hence, the network 2030 may include links using technologies such as Ethernet, 802.11, worldwide interoperability for microwave access (WiMAX), 2G/3G/4G/5G mobile communications protocols, InfiniBand, PCI Express Advanced Switching, etc. Other networking protocols used on the network 2030 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for improving image quality without increasing dose of contrast agent, the method comprising:
(a) receiving an input image comprising a pre-contrast image and a full-dose image, wherein the pre-contrast image is a volumetric medical image of a subject acquired without administering contrast agent and the full-dose image is a volumetric image of the subject acquired with standard dose of contrast agent;
(b) selecting a path from a plurality of paths to process the input image, wherein the path comprises at least a first model trained to predict a contrast-enhanced image, and a second model trained to denoise an image and wherein the first model and the second model are arranged in a predetermined order to process the input image; and
(c) generating a predicted image by processing the input image using the path selected in (b), wherein the predicted image has an image quality improved over the input image.

2. The computer-implemented method of claim 1, wherein the path further comprises a third model trained to improve a resolution of an image.

3. The computer-implemented method of claim 2, wherein each of the plurality of paths comprises two or more of the first model, the second model and the third model arranged in a predetermined order.

4. The computer-implemented method of claim 1, wherein the input image further comprises one or more reformatted volumetric medical images of the pre-contrast image or the full-dose image.

5. The computer-implemented method of claim 4, wherein the one or more reformatted volumetric medical images are generated by reformatting the pre-contrast image or the full-dose image in one or more orientations.

6. The computer-implemented method of claim 5, wherein the one or more orientations include at least one orientation that is not in a direction of a scanning plane.

7. The computer-implemented method of claim 1, wherein at least one of the plurality of paths comprises two of the second models to denoise the pre-contrast image and the full-dose image respectively.

8. The computer-implemented method of claim 1, wherein the pre-contrast image or the full-dose image is acquired using a transforming magnetic resonance (MR) device.

9. The computer-implemented method of claim 8, wherein the input image comprises different contrast-weighted images acquired using different pulse sequences.

10. The computer-implemented method of claim 9, wherein the different contrast-weighted images comprise two or more selected from the group consisting of T1-weighted (T1), T2-weighted (T2), proton density (PD) or Fluid Attenuation by Inversion Recovery (FLAIR).

11. The computer-implemented method of claim 10, wherein at least one of the plurality of paths comprise a multi-contrast branched architecture.

12. The computer-implemented method of claim 11, wherein the multi-contrast branched architecture comprises multiple branches and wherein inputs to the multiple branches are different in at least one of dose of contrast agent and pulse sequence.

13. The computer-implemented method of claim 12, wherein each of the multiple branches comprises a first model trained to learn features of the respective input image.

14. The computer-implemented method of claim 12, wherein a plurality of synthesized images generated by the multiple branches are aggregated and is further processed by a trained model generate a final output image.

15. A non-transitory computer-readable storage medium including instructions that, when executed by one or more processors, cause the one or more processors to perform operations comprising:
(a) receiving an input image comprising a pre-contrast image and a full-dose image, wherein the pre-contrast image is a volumetric medical image of a subject acquired without administering contrast agent and the full-dose image is a volumetric image of the subject acquired with standard dose of contrast agent;
(b) selecting a path from a plurality of paths to process the input image, wherein the path comprises at least a first model trained to predict a contrast-enhanced image, and a second model trained to denoise an image and wherein the first model and the second model are arranged in a predetermined order to process the input image; and
(c) generating a predicted image by processing the input image using the path selected in (b), wherein the predicted image has an image quality improved over the input image.

16. The non-transitory computer-readable storage medium of claim 15, wherein the path further comprises a third model trained to improve a resolution of an image.

17. The non-transitory computer-readable storage medium of claim 16, wherein each of the plurality of paths comprises two or more of the first model, the second model and the third model arranged in a predetermined order.

18. The non-transitory computer-readable storage medium of claim 15, wherein the input image further comprises one or more reformatted volumetric medical images of the pre-contrast image or the full-dose image.

19. The non-transitory computer-readable storage medium of claim 18, wherein the one or more reformatted volumetric medical images are generated by reformatting the pre-contrast image or the full-dose image in one or more orientations.

20. The non-transitory computer-readable storage medium of claim 19, wherein the one or more orientations include at least one orientation that is not in a direction of a scanning plane.
